(19) United States
(12) Patent Application Publication
(10) Pub. No.: US 2016/0051697 A1
(43) Pub. Date: Feb. 25, 2016

(54) NANODELIVERY DEVICE FOR THERAPEUTIC LOADING OF CIRCULATING ERYTHROCYTES

(71) Applicant: THE BOARD OF REGENTS FOR OKLAHOMA STATE UNIVERSITY, Stillwater, OK (US)

(72) Inventors: CAREY N. POPE, Stillwater, OK (US); ASHISH RANJAN, STILLWATER, OK (US); JING LIU POPE, STILLWATER, OK (US); STEVE HARTSON, STILLWATER, OK (US); JOSHUA RAMSEY, STILLWATER, OK (US)

(21) Appl. No.: 14/680,862

(22) Filed: Apr. 7, 2015

Related U.S. Application Data
(60) Provisional application No. 61/975,993, filed on Apr. 7, 2014.

(51) Int. Cl. A61K 47/48; A61K 38/46

(52) U.S. Cl. CPC A61K 47/48907; A61K 47/48692; A61K 47/48561; A61K 47/4843; A61K 47/48215; A61K 38/465; A61K 47/48246; C12Y 301/01008

(57) ABSTRACT

According to one embodiment, a person's own RBCs can be recruited as secondary bioscavenger carriers in vivo using a nanopolymer-BChE complex, with an affinity ligand (antibody or peptide) for selective targeting to the RBCs and a cell-penetrating peptide for uptake into the RBCs. A general approach according to an embodiment involves parenteral administration of the nanodevice to gain access to the systemic circulation, which then seeks out and attaches to the person's RBCs, followed by transport into the RBCs (to minimize clearance from the circulation), leading to long-term circulation of the bioscavenger enzymes and thus protection against intoxication.

### CROSS REFERENCE TO RELATED APPLICATIONS

This application claims the benefit of U.S. Provisional Patent Application Ser. No. 61/975,993 filed on Apr. 7, 2014, and incorporates said provisional application by reference into this document as if fully set out at this point.

### TECHNICAL FIELD

This disclosure relates to the use of red blood cells (RBCs) as biocompatible carriers for agents of interest. In particular, the invention relates to RBCs loaded in situ via an RBC-targeted nanodelivery device which comprises the agents of interest, and methods of using the same.

### BACKGROUND

Organophosphorus anticholinesterases (“OPs”) are among the most toxic of synthetic chemicals. Even non-lethal intoxications can lead to long term, persistent health problems. The pronounced toxicity of OPs is based on their potent effects on the enzyme acetylcholinesterase (AChE). An OP binds covalently to the active site serine in AChE, leading to impaired degradation of the neurotransmitter acetylcholine. Extensive ACNE inhibition leads to elevation of acetylcholine at cholinergic synapses and prolonged stimulation of cholinergic receptors in a variety of tissues and organs. The effects of OPs on AChE led to their widespread use as insecticides, among other applications. Unfortunately their potent effects have been misused in chemical warfare and chemical terrorism.

OP intoxication can be expressed as a variety of signs and symptoms including respiratory dysfunction, tremors and seizures, cardiovascular abnormalities, increased parasympathetic-mediated secretions, and others. OP intoxication is routinely treated with atropine (to block muscarinic cholinergic receptor activation), an oxime to reactivate the inhibited AChE and in some cases a benzodiazepine to minimize seizures and convulsions. While this strategy is effective, it has marked limitations based on the short term drug effects against the long-term cholinergic system disruption, the inability of oximes to reactivate “aged” AChE, the difficulty of oximes to pass the blood brain barrier to reactivate AChE in the central nervous system, and others. There continues to be intense interest in the development of effective countermeasures against OP intoxication.

One approach to prophylaxis of OP intoxication involves pre-administration of another AChE inhibitor. A more reversible inhibitor, (e.g., the carbamate inhibitor pyridostigmine) can be used to “occupy” the active site region of AChE, and thereby protect the enzyme from binding to and phosphorylation by an OP. If exposure to an OP occurs while the enzyme is bound by a short-lasting inhibitor, the reactive OP molecules will bind to other non-AChE proteins, resulting in net protection from OP toxicity. This prophylactic strategy depends on inhibition of a proportion of AChE molecules (about 30%), which can be tolerated without influencing cholinergic signaling. Those same pre-inhibited enzymes can become catalytically functional within a short period should OP exposure occur. A difficulty with this approach is the short duration of enzyme protection, requiring repeated dosing.

Another approach involves the use of bioscavengers that act as “sponges” for binding/inactivating toxicants in the circulation system. Systemic administration of bioscavenger enzymes has been shown to sequester OP molecules, inactivating them before they could reach critical AChE molecules in the brain and peripheral tissues. Several types of scavenger enzymes have been evaluated for protection against OP intoxication including carboxylesterases, butyrylcholinesterase (BChE), and AChE. In sufficient quantities, administration of these stoichiometric binding proteins can minimize tissue AChE inhibition following OP exposure. Importantly, this approach prevents some of the most debilitating consequences of OP intoxication, e.g., seizures and neuropathology.

Difficulties in the prophylactic use of bioscavenger enzymes include the large amounts of enzyme required and the rapid clearance of the enzyme from the circulation. While the duration of prophylaxis with native enzyme is prolonged relative to the use of a short-term inhibitor, native enzyme is cleared from the circulation within a matter of days. Intramuscular (im) administration of human BChE in mice (13 mg/kg, about 0.3 mg) led to peak blood BChE activities at about 10-12 hours, with only about 25% of peak levels still present at 70 hours. Following either im or intraperitoneal (ip) administration of 0.1-3 mg of human BChE in mice, marked elevation of circulating BChE enzyme activity was noted, peaking at 12-24 hours after administration, but levels were back to near baseline by 120 hours. Pharmacokinetics of human BChE in mice and guinea pigs have been reported following im and ip administration, long-term stability (at least 2 years) of lyophilized enzyme, and complete, sign-free survival in guinea pigs given an LD₅₀ dosage of the nerve agents VX and soman. Using higher im dosages of human BChE (up to 60 mg/kg), peak enzyme levels were noted at around 24 hours with substantial levels of circulating BChE activity still noted for at least four days after dosing with the highest dosage. No overt physiological or behavioral signs, changes in serum chemistry, or tissue histopathology were reported following high dose BChE administration. A recent study reported that intravenous (IV) administration of 30 mg/kg human BChE led to marked elevation of blood BChE activity that remained elevated for 100 hours. Together with a number of other publications, these findings show that parenteral administration of human BChE can lead to marked elevation of circulating enzyme levels and protection against the toxicity of a number of potent OP nerve agents, with no evidence of any adverse reactions to the enzyme.

Encapsulation within polymer matrices or modified with polymers has been shown to extend protein circulation times in vivo. For example, many studies have investigated encapsulating proteins within poly(ethylene glycol) (PEG)-PLGA microspheres or PEG-PLGA nanoparticles for controlled release of protein. Other approaches have been used to extend the circulatory lifetime of the protein itself, as well as other drugs and nanoparticles. PEG has been shown to improve biodistribution by preventing immune recognition and clearance by the reticuloendothelial system.

Recently it was demonstrated that a grafted copolymer composed of poly(L-lysine) (PLL) and PEG could be used to encapsulate BChE and improve significantly its bioavailability in vivo compared to the free protein alone. Depending on the source, BChE has an isoelectric point between 4.2-4.9, giving it a negative charge at pH 7. Lysine amine groups that comprise PLL give the polymer an isoelectric point of 10.8 and a highly cationic charge at pH 7. Consequently, mixing BChE and PLL at neutral pH will result in the spontaneous formation of PLL/BChE nanoparticles. The addition of the PEG polymer prevents many of the problems that would be associated with introducing highly charged foreign particles in vivo. Using this type of polymer to electrostatically encapsulate BChE can provide benefits in terms of improved biodistribution and bioavailability, flexible targeting, and reduced reticuloendothelial uptake.

RBCs have been used as biocompatible vectors for drug, peptide and protein delivery. The general approach is removal of RBCs, loading of the “payload” into lysed cells, resealing, and then reinfusion of the resealed RBCs. For example, bacterial asparaginase has been loaded into RBCs and used to treat acute lymphoblastic anemia. Parenteral delivery of the free enzyme led to hypersensitivity reaction and other adverse responses including coagulation abnormalities and immunosuppression, while the RBC-encapsulated enzyme did not. Manipulation of the RBC membranes during encapsulation dramatically alters RBC contents and function, however. Therefore, while the use of RBCs packaged with a therapeutic payload and resealed outside the body may have many applications, this approach poses many problems as well.

In summary, for all of its promise, the bioscavenger approach suffers from, among others, a relatively rapid clearance from the body and therefore need for large doses of purified enzyme. If the circulation time of bioscavengers could be prolonged, that would minimize enzyme need and increase the duration of protection.

As such, there has been a real need for a system and method that provides a more effective way to utilize bioscavengers in the prophylactic and real-time treatment of individuals against exposure to OPs. Accordingly, it should now be recognized, as was recognized by the present inventors, that there exists, and has existed for some time, a very real need for an invention that would address and solve the above-described problems.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

Other embodiments and variations are certainly possible within the scope of the instant invention and can readily be formulated by those of ordinary skill in the art based on the disclosure herein.

Parenteral administration of butyrylcholinesterase (BChE) is an effective prophylactic strategy to counteract organophosphorus anticholinesterase (OP) toxicity. However, as is the case for many other therapeutic agents, its efficacy is hampered by rapid clearance of the enzyme from the circulation. Red blood cells (RBCs) have been utilized as carriers of payloads e.g. for the enzyme asparaginase. However, the techniques used to load the RBCs involved RBC collection, lysing, loading, and then resealing the RBCs, a laborious undertaking. Further, resealing of RBCs is not always successful and the RBCs may become non-viable and/or may “leak” the payload prematurely.

The present invention provides novel methods of using RBCs as secondary drug delivery agents. The methods do not require hands-on manipulation of the RBCs. According to the invention, in one aspect what is provided is a polymeric nanoparticle comprising i) an agent of interest that binds at least one ligand of interest and ii) a targeting moiety that specifically (or at least selectively) targets RBCs. The at least one ligand of interest that is bound by the agent of interest may be a noxious compound. Administration of the NP to a subject in a manner that permits contact between the NPs and RBCs of the subject results in binding of the NPs to RBCs via the targeting moiety. The agent of interest is thus sequestered at the RBC surface, traverses the circulatory system with the RBC, and is free to interact with other molecules it encounters during circulation. If the agent encounters the at least one ligand, it binds the ligand and thus removes it from circulation, preventing its activity. The RBC-NP complex thus can, in some aspects, serve as a scavenger complex to remove unwanted substances from circulation.

In other aspects, the NPs also comprise at least one RBC penetrating moiety that, upon contact with an RBC (mediated by the targeting moiety), mediates entry of the NP into the RBC. Administration of the NPs to a subject in a manner that permits contact between the NPs and RBCs of the subject thus results in entry of the NP into an RBC, thereby loading the RBC with the associated agent. The method is much less invasive than collecting RBCs from a donor and readministering them to a subject, and is less damaging to the RBCs themselves, yet preserves the advantages of sustained drug delivery using RBCs since the agent of interest is sequestered within RBCs. As the red blood cells circulate for 90-120 days, once internalized the encapsulated scavenger molecules can afford “on demand” protection.

In both of these aspects, the NP components are degraded along with their associated RBCs, e.g. in the normal apoptotic pathways mediated by the reticuloendothelial system. If the NPs are internalized within RBCs, there is a slow breakdown of the polymer and release of the free enzyme within the cytosol. If the nanodevice does not enter into RBCs, NPs attached to the cell surface are also degraded over time, releasing the associated agent directly into the circulation.

The foregoing has outlined in broad terms the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1. Schematic illustration of grafted copolymer synthesis.

FIG. 2 shows a sample NMR spectrum of 4-15 kDa PLL grafted with 2 and 5 kDa PEG.

FIG. 3 shows an exemplary grafted copolymer library.

FIG. 4 shows a schematic representation of BChE nanoparticle synthesis.

FIG. 5A-C shows lab results of particle synthesis confirmed by dynamic light scattering (DLS) of A, BChE alone; B, BChE associated with copolymer nanoparticles; and C, polyacrylamide gel electrophoresis (PAGE) of altered migration of encapsulated vs non-encapsulated enzyme. Note that in the lanes 4-6 from left to right, the major protein band begins to migrate less as more copolymer is added to the enzyme (5C).

FIG. 6. Encapsulation of all nine combinations of polymer molecular weight and PEG grafting ratios. PAGE was conducted with (left-right) free enzyme (lane 1), crosslinked enzyme (lane 2), and then 0.6, 1, 2, 7, 10 and 14:1 copolymer to enzyme ratio preps (lanes 3-8).

FIG. 7. PAGE separation of encapsulated BChE nanoparticles (medium molecular weight, 10:1 grafting ratio) with various copolymer to BChE ratios. Boxed areas show positive “hits’ using mass spectroscopy fingerprinting of excised gel bands.

FIG. 8A-C. BChE nanoparticle analysis by electron microscopy. A, scanning electron microscopy (SEM); B, transmission electron microscopy (TEM); C, graphical representation of particle size, with 165 particles counted and an average size of about 70 nm.

FIG. 9A and B shows the average enzyme activity of 3-4 different preparations of NPs using medium molecular weight (A, left panel) and low molecular weight (B, right panel) polymer, both with 10:1 grafting ratio. As shown in this figure, there is relatively minimal loss of activity compared to the original starting enzyme.

FIG. 10A and B shows dialysis of either the free enzyme, low molecular weight or medium molecular weight NPs (both with 10:1 grafting ratio, 7:1 copolymer:enzyme ratio). There was a “size’ dependent difference in rate of diffusion, suggesting differences in passing through the 300 kDa membrane. A, pass-through; B, retentate.

FIG. 11 A and B shows stability of the respective nanoparticles (A, low vs B, medium molecular weight, 10:1 grafting ratio) at 37° C. for up to 28 days. The NPs appeared very stable under these conditions. (Stability tests of enzyme activity of NPs incubated at 37° C. in PBS containing 0.5 mg/ml bovine serum albumin.)

FIG. 12 shows the scheme for targeting NPs synthesis. A maleimide terminal group is used on the PEG residues for attachment via disulfide linkage with a targeting ligand. For the preclinical assessments, these targeting ligands include monoclonal against glycophorin A, the single chain construct (scFv), or the high affinity peptide ERY1. For practical application, analogs of these targeting human glycophorin A will be used.

FIG. 13 shows flow cytometric evaluation of binding of the ERY1-liganded medium molecular weight, 10:1 grafting ratio, 7:1 copolymer:enzyme ratio NPs to mouse RBCs, in this case with fluorescent-tagged BSA in place of BChE. The shift in fluorescence intensity in NPs without (top panel) or with (middle panel) ERY1 attachment suggests increased binding to mouse RBCs with the ligand.

FIG. 14A and B shows interaction of the organophosphate paraoxon with free and nano-encapsulated butyrylcholinesterase. Medium molecular weight (MMW) 10:1 grafting ratio, 7:1 co-polymer-enzyme ratio (MMW10, 7:1) is shown on the top panel (A) and low molecular weight (LMW), 10:1 grafting ratio, 7:1 copolymer-enzyme ratio (LMW10, 7:1) is shown on the bottom (B).

FIG. 15 A-B. Paraoxon sequestration assays. Medium molecular weight (MMW10, 7:1) NPs (A, top panel) or low molecular weight (LMW10, 7:1) NPs (B, bottom panel) were pre-incubated with paraoxon (40 nM final concentration) for 20 minutes at 37° C. Pig liver carboxyl esterase was then added and allowed to incubate for an additional 20 minutes, and then substrate was added to measure residual carboxylesterase activity.

FIG. 16 shows the data from FIG. 15 plotted as log of the NP dilution vs percent protection of the carboxylesterase from inhibition by paraoxon.

FIG. 17. Schematic illustration of a RBC-targeted nanoparticle and attachment to RBC.

FIG. 18. Schematic illustration of a RBC-targeted nanoparticle and internalization within RBC, and subsequent release from RBC.

FIG. 19. Schematic illustration of Ter-119, single chain antibody against GPA is specific to erythroid cells (RBCs).

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

Provided herein are nanoparticles (NPs) which serve as nanodelivery devices. The NPs comprise i) at least one agent of interest associated with a polymeric matrix; ii) an RBC targeting moiety; and, in some embodiments, iii) a cell penetrating moiety. When the NP comes into contact with an RBC, the targeting moiety binds to the RBC surface, thereby externally sequestering the NP to the RBC. Such copolymer NPs bind to RBCs without internalization and act, for example, as nanoscavengers for a compound that is bound by the at least one agent of interest. If a cell penetrating moiety is also present in the NP, it is also brought into proximity to the RBC surface by the action of the targeting moiety where it mediates entry of the nanoparticle (together with the encapsulated agent of interest) into the interior of the RBC. RBC internalized NPS are released into circulation only upon destruction of the RBC.

Both of these in situ RBC loading aspects of the invention are less invasive than collecting RBCs from a donor and readministering them to a subject, and are less damaging to the RBCs themselves. Disruption of the RBC is minimal, and the RBC continues to circulate and otherwise function normally, completing its lifespan (e.g. whatever remains of its usual 90-120 day life span). In effect, a subject's own RBCs are recruited and “piggy-back” transport by RBCs is employed to prolong circulation of an agent of interest.

If the NP is not internalized, it is free to carry out its intended activity whenever it encounters a ligand in the environment. If the NP is internalized, it can bind to unwanted substances that can readily pass through the cell membrane and into the red blood cell, and/or upon senescence of the RBC, the nanoparticle is released into circulation and the agent remains free to carry out its intended activity. Since a given population of RBCs contains RBCs of many different degrees of maturity, the release of agent into circulation begins soon after treatment as the oldest RBCs are degraded, and continues until those which were the youngest at the time of administration are degraded, e.g. for a maximum of about 120 days. In addition, it is likely that some benefits will accrue even if some nanoparticles do not attach to RBCs since the agents they carry will be active immediately and/or if some nanoparticles attach to RBCs but are not internalized even if the NPs contain a RBC penetrating agent (the agents they carry will still circulate and be active while bound to an RBC). In fact, if externally attached NPs become detached from the RBC, e.g. by proteolysis, hydrolysis, mechanical shear, etc., they can still perform their intended activity until they themselves are degraded.

Thus, according to some aspects, red blood cells (RBCs) are recruited to serve as carriers for agents of interest such as drugs, peptides and enzymes. An embodiment utilizes cationic copolymer nanoparticles (NPs) to facilitate delivery of encapsulated (electronically complexed) agents such as BChE to RBCs for prolonged circulation. When the exemplary protein recombinant human BChE was used as the agent of interest, the NPs proved to be stable for weeks at 37° C. When attached to the NPs, a monoclonal antibody, a single chain antibody to the erythroid cell-specific membrane protein glycophorin A (anti-GPA), or a high affinity peptide that binds to glycophorin A allows selective targeting to RBCs, while a cell-penetrating peptide (CPP) facilitates entry into cells. Once bound to/internalized into RBCs, the NPs circulate for prolonged periods of time providing long-term protection, e.g. for weeks or even months, since an RBC lifetime typically ranges from about 90 to about 120 days.

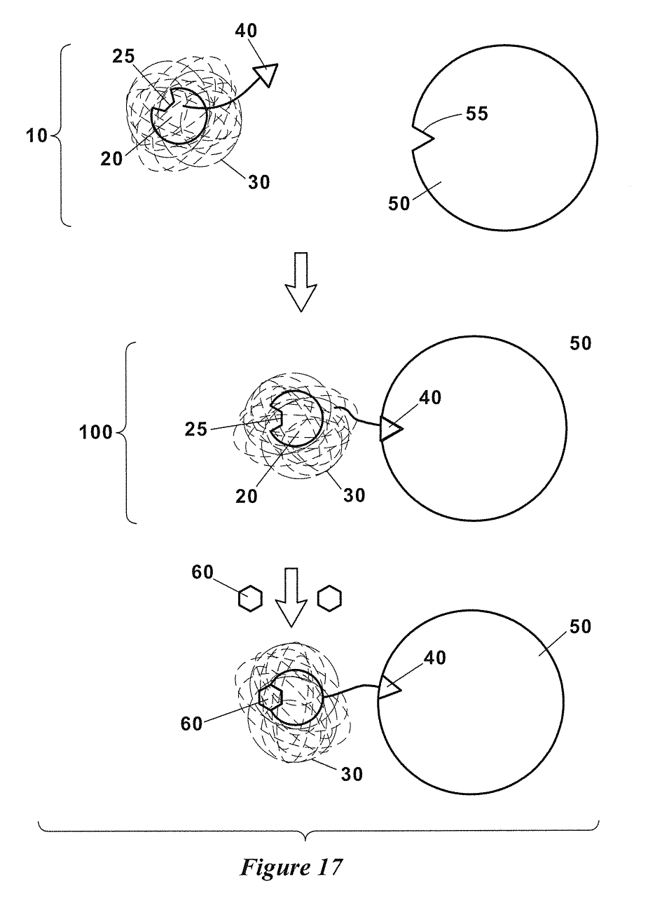

A schematic illustration of a first aspect of the invention is provided in FIG. 17. In FIG. 17, nanoparticle 10 comprises active agent 20 permeably encapsulated in copolymers 30. Active agent 20 comprises ligand binding site 25 which is specific or selective for binding to a ligand of interest. Active agent 20 is “permeably encapsulated” because, while surrounded or encapsulated by copolymers 30, molecules, especially small molecules such a ligand of interest, can penetrate the capsule and access ligand binding site 25. NP 10 also comprises RBC targeting moiety 40 which is capable of binding specifically or selectively to binding site 55 of RBC 50. When NP 10 encounters RBC 50, targeting moiety 40 binds to binding site 55 of RBC 50, forming RBC-NP complex 100. When a compound (e.g. ligand of interest 60) in the environment (e.g. in the circulatory system) makes contact with ligand binding site 25, binding and sequestration of ligand of interest 60 occurs, removing ligand of interest 60 from circulation. NP-RBC complex 100 thus acts as a mobile or peripatetic scavenging device for ligand of interest 60 within the circulatory system.

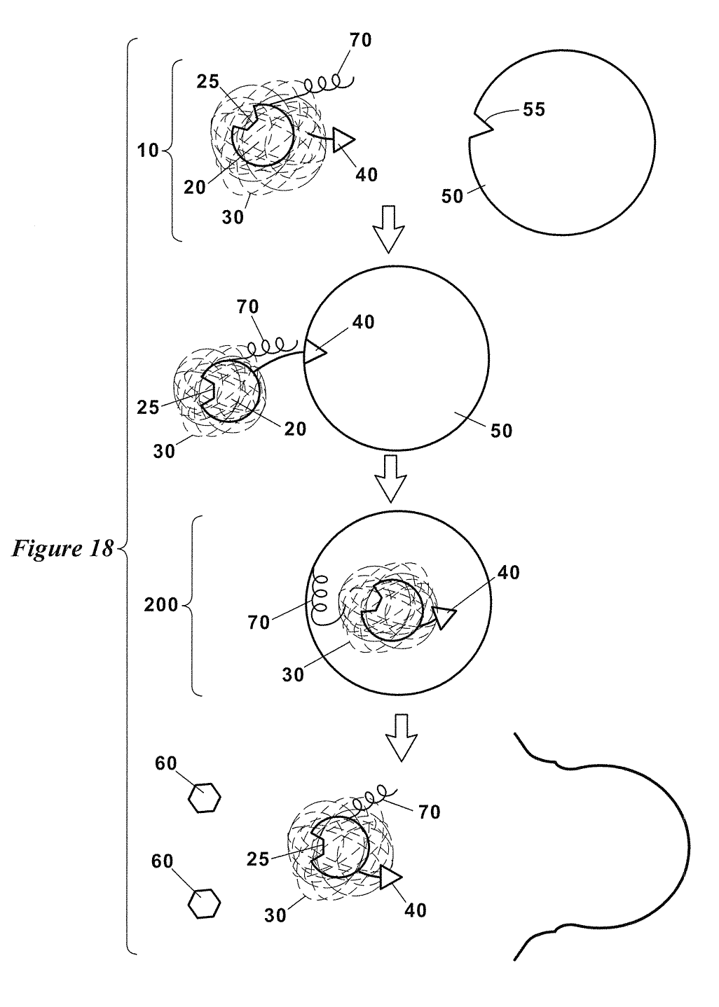

In another aspect, schematically illustrated in FIG. 18, NP 10 comprises, in addition to active agent 20 with ligand binding site 25 and RBC targeting moiety 40, penetrating moiety 70. In this aspect, when RBC targeting moiety 40 binds to binding site 55 of RBC 50, penetrating moiety 70 is brought into proximity to RBC 50, and exerts its action of internalizing NP 10 within RBC 50, forming NP-internalized RBC 200. NP-internalized RBC 200 carries NP 10 as it traverses the circulatory system, allowing binding to unwanted substances as they pass into the RBC. Upon natural destruction of NP-internalized RBC 200 (e.g. via apoptosis), NP 10 is released into circulation and exerts its activity, e.g. binding of ligand 60 to binding site 25. As shown in FIG. 18, after release from the RBC, NP 10 still retains penetrating moiety 70 and targeting moiety 40. However, it is possible that these two moieties will have been removed during entry into the RBC, and/or during internalization within the RBC and/or during the apoptotic activity that eliminates the RBC. Further, it is possible that only active agent 20 is released upon apoptosis, i.e. that one or more of targeting moiety 40, penetrating moiety 70 or polymer 30 may already be degraded within the RBC prior to release.

According to aspects of the invention, polymers are utilized to encapsulate an agent of interest that is to be associated with an RBC. In some aspects, the polymers are copolymers, i.e. they are obtained by copolymerization of two or more monomer species. In some aspects, the copolymers are grafted copolymers, i.e. the copolymers are branched copolymers in which the side chains are structurally distinct from the main chain. For example, the main chain and the side chains may be composed of distinct homopolymers, or alternatively, the individual chains of a grafted copolymer may be homopolymers or copolymers. Various alternating copolymers and block copolymers are also encompassed, as are e.g. diblock copolymers with “A-B” alternating copolymer side chains (which may also technically be termed grafted copolymers).

Many types of monomers may be utilized to make the polymers/copolymers that are employed in the invention. Typically, the components are biocompatible and can function in intimate contact with living tissue without damaging the tissue. In some aspects, the components are also biodegradable. Exemplary components include but are not limited to: poly(esters) based on polylactide (PLA), polyglycolide (PGA), polycaprolactone (PCL); various poly(saccharide)s e.g., starch, cellulose (and derivatives thereof, including without limitation: cellulose acetate phthalate, cellulose acetate trimellitate, hydroxypropyl methylcellulose phthalate, and carboxymethylethylcellulose), chitosan, chitin, dextrans, and lignins; biodegradable copolymers with glycolide and e-caprolactone; amino acid polymers such as poly-lysine, poly-ornithine, poly-arginine, poly-glutamic acid, poly-proline, poly-tyrosine, poly-alanine, etc.; various vinyl derivatives such as polyvinyl acetate phthalate; acrylates such as co-polymerized methacrylic acid/methacrylic acid methyl esters; and the polyether polyethylene glycol (PEG) of various forms and molecular weights; etc., and derivatives thereof.

Copolymers comprising one or more PEGs may be used to form the NPs. PEGs are available over a wide range of molecular weights e.g. from 300 g/mol to 10,000,000 g/mol. Different forms of PEG are also available, including monofunctional methyl ether PEG, or methoxypoly(ethylene glycol), abbreviated mPEG. The PEGS may be monodisperse or polydisperse, and may be of different geometries such as branched (having three to ten PEG chains emanating from a central core group), star (having 10 to 100 PEG chains emanating from a central core group) and comb (having multiple PEG chains normally grafted onto a polymer backbone). The polymers/copolymers that are used in the practice may be formed from any suitable component or combination of components, such as those disclosed herein.

In some aspects, the polymeric subunits comprise one or more reactive groups that are capable of functionalization, i.e. are capable of reacting with, usually binding to or covalently bonding with, a compound of interest. Compounds of interest include but are not limited to: linking molecules and moieties; targeting moieties; penetrating moieties, etc. as described in detail below.

In some aspects, the copolymers are grafted copolymers comprised of poly (L-lysine) (PLL) and PEG, formed, for example, from a 50/50 mixture of 2 kDa and 5 kDa mPEG reacted with poly(L-lysine) (PLL) polymers with molecular weight ranges 4-15 kDa, 15-30 kDa, and 30-70 kDa. Lysine amino groups have an isoelectric point of about 11 and they are positively charged at pH 7. In an exemplary embodiment, the agent of interest is the enzyme BChE, which has an isoelectric point of about 4.5 and is negatively charged at pH 7. When polymers of PLL come into contact with BChE at about pH 7, self assembly of PLL/BChE nanoparticles occurs spontaneously due to the differences in charge. Inclusion of PEG in the copolymers promotes reduced reticuloendothelial uptake and reduced immunogenicity.

According to the invention, a nanoparticle is provided which comprises at least one agent of interest (that exerts an activity of interest) encapsulated by one or more polymers, usually a copolymer, and more usually a grafted copolymer. Exemplary agents of interest which can be so-encapsulated include, without limitation: proteins [for example enzymes such as asparaginase, e.g. to treat acute lymphoblastic leukemia; BChE, e.g. to treat or prevent poisoning or intoxication such as with OP, etc.], peptides, nucleic acids, various drugs [e.g. an antibiotic that is rapidly cleared or that is needed to protect infectious agents that target RBCs] and small molecules, scavenging agents (agents that bind to and retain at least one ligand of interest), metals and metal complexes, vitamins, cofactors, etc.

This embodiment has the potential to have a profound impact on chemical warfare agent science. Effective, practical approaches to protection against nerve agent intoxication have been problematic. This embodiment could be revolutionary in providing sustained, prolonged levels of bioscavengers in the circulation following a single administration. Attached, but in particular, internalized into circulating RBCs, BChE-nanoparticles could circulate for months, being shielded from proteolytic attack and immune surveillance. The successful implementation of this strategy involving nano-encapsulation of a therapeutic/prophylactic agent, affinity targeting to selected cell types including non-RBC cell types utilizing different targeting ligands, and subsequent internalization of the nanodevice via cell penetrating peptide mechanisms, all mediated by a nanodevice given parenterally, could yield advances in treatment of other disorders and conditions that would benefit by prolonged circulation of the therapeutic or prophylactic agent. Examples of such disorders and conditions include but are not limited to: disorders of immune cells, infections that target circulating blood cells, diseases of/related to erythrocytes (e.g., sickle cell anemia, malaria).

In some aspects, the agent of interest is the enzyme BChE. The preparation of enzymes is known in the art. An exemplary protocol for providing BChE for use in the present invention is as follows: HEK 293 cells are transduced with an adeno-associated viral vector (AAV-BChE plasmid) or transfected with a standard expression plasmid encoding BChE. Three days after transduction or transfection, human BChE is isolated from culture supernatants by chromatography on procainamide-Sepharose gel followed by ion-exchange chromatography. Purification yields one major band on SDS polyacrylamide gels. To determine the final molar concentration of enzyme, active sites are titrated with diisopropylfluorophosphate as described previouslyl^{[47]}. Enzyme kinetics are tested with [³H]acetylcholine across a wide range of substrate concentrations and comparable experiments are performed with acetyl- and butyrylthiocholine substrates in spectrophotometric assays adapted for plate reader^{[48]}. For estimates of Vₘₐₓ, Kₘ, K_{SS}, and “b”, kinetic data are fitted to the appropriately modified Michaelis-Menten equation with Sigma Plot as previously described^{[49,50]}. For more rapid or higher yield human BChE purification, an immunoaffinity chromatography procedure using high affinity anti-BChE monoclonal antibody as a capture reagent can be employed^{[52]}.

In some aspects, the activity of interest that is carried out by the agent of interest is binding to a ligand, especially to a ligand which is undesirable. In so doing, the agent of interest removes the ligand from circulation and prevents its undesirable or noxious activity. In some aspects, the agent of interest competes with another molecule in the body for binding to the ligand. Binding between the agent and the ligand may be of any suitable type, e.g. binding may be irreversible or reversible, but if binding is reversible, the Km of binding is sufficient to decrease the effective concentration of free ligand in circulation so that untoward effects which would otherwise occur due to the activity of the ligand are prevented or at least decreased. In such aspects, the agent of interest serves as a scavenger of the unwanted ligand. However, other aspects of the invention provide agents with other activities, such as various desirable enzyme activities (outside or within RBCs), delivery of a medicament that is attached to but proteolytically cleaved from the agent of interest upon exposure to the circulatory system, etc. Any agent with a desired activity for which it may be useful to provide a vehicle for circulating in the blood may be encapsulated and attached and/or internalized in RBCs as described herein.

Exemplary ligands that may be bound by an agent of interest as described herein include but are not limited to: various small molecules and drugs (e.g. when a drug overdose occurs), poisons, chemical warfare agents, gases, OP, etc.

The nanoparticles described herein comprise at least one targeting moiety with an affinity for binding to the surface of RBCs, e.g. to a binding or targeting (or targeted) site. The targeting moiety may be, for example, a natural or artificial ligand, a substrate, an enzyme or portion thereof, an antibody (polyclonal or monoclonal), such as single chain antibodies, a high affinity peptide, a diabody, an aptamer, etc.

In some aspects, the targeting moiety is specific or selective for one of a variety of RBC surface proteins which serve as selective docking sites for nanodelivery devices, e.g., band 3, CD47, VLA-4, glycophorin A and others. Band 3 is a major integral protein on the erythrocyte plasma membrane, with approximately a million copies in each cell. Band 3 interacts with several proteins (e.g., ankyrin, protein 4.1) serving as the primary anchoring site for cytoskeletal elements. Through these interactions, band 3 plays an important role in overall organization of the RBC membrane and flexibility/rigidity of the cell and is a targeting site on the RBC membrane.

A protein that binds to band 3 with utility as a RBC targeting site for the nanodevices disclosed herein is glycophorin A (GPA). GPA is a highly conserved, erythroid-specific transmembrane protein. It is highly expressed on the membrane (approximately the same level as band 3). Surface charge is not affected in GPA-deficient RBCs and they appear to maintain normal shape, suggesting that GPA may not be critical for RBC stability. The Wright b antigen (Wrb) is located on glycophorin A, and this protein also functions as the receptor for the malaria parasite Plasmodium falciparum.

Antibodies to GPA have been used as specific markers for erythroid cells in experimental, epidemiological and even forensic studies. Importantly, GPA has been successfully used as a docking site for RBCs. It has been reported that mouse RBCs can be effectively labeled with a single chain antibody to GPA (scFv Ter-119) and that a fusion product of scFv Ter-119 with a complement-associated protein selectively bound to mouse RBCs in vivo. Remarkably, levels of the fusion complex were relatively stable in the circulation during the observation period (1-4 days after dosing). For example, it has been reported that when scFv Ter-119 was fused with another protein (prourokinase), bound to mouse RBCs ex vivo, and cells injected into mice, circulating enzyme activity was markedly prolonged compared to mice treated with enzyme alone. About 95% of the circulating enzyme remained bound to RBCs. scFv Ter-119 fused to mutant urokinase extensively bound mouse RBCs (over 40% of the injected dose, with an estimated 30,000 copies per cell). The cells exhibited no change in aggregation, hemolysis, or uptake by the reticuloendothelial system. Another single chain anti-GPA (10F7 scFv) was used to enhance binding of engineered forms of eryrthropoietin to erythroid cells. Recently, synthesis of antigen constructs to target erythrocyte cell surfaces either with an erythrocyte-binding peptide or with an antibody fragment was reported, both targeting GPA. Thus, a number of studies suggest that GPA is a selective targeting molecule for eyrthroid cells that can serve as a selective docking site for RBC targeting. As noted, effective targeting of scFv Ter-119-fused proteins to the RBC membrane has been achieved in several studies, which markedly prolongs circulation of the fused proteins.

In some aspects, scFv Ter-119 is used in the practice of the present invention for proof of concept and preclinical studies in mice. For practical application, an analog of scFv targeting human glycophorin A (which is a membrane antigen expressed exclusively on erythroid cells) is used. Single scFv peptides may be used; however, in some aspects, what is used is a polypeptide comprising multiple copies of the scFv, i.e. the sequence is duplicated, triplicated, etc. to create a single polypeptide with two, three or more full scFv sequences.

In some aspects, the targeting moiety is a monoclonal antibody against glycophorin A, a scFv Ter-119 (a single chain antibody for mouse glycophorin A or an analogous scFv against human glycophorin A), or ERY-1 (a 12-amino acid peptide from a phage-display library that is known to bind tightly to glycophorin A, and for practical application the analog selected against human glycophorin A).

In some aspects, the NPs of the invention also comprise a penetrating (penetration, internalizing, internalization, etc.) moiety that facilitates (mediates, causes, etc.) transfer or entry of the NPs into the interior of RBCs to which they are attached. Many examples of such molecules are known in the art, including but not limited to: various peptide sequences such as cell-penetrating peptides (CPPs), e.g., low molecular weight protamine, penetratin, Tat protein, transportan, etc.

In some aspects, the penetrating moiety is a CPP. CPPs are short peptides, typically less than 20 amino acids, that facilitate cellular uptake of various molecular cargo (from nanosize particles to small chemical molecules to large particles, including peptides, proteins, oligonucleotides, plasmids, and nanoparticles). The “cargo” is associated with the peptides either through chemical linkage via covalent bonds or through non-covalent interactions. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Their function is to deliver the cargo into cells, a process that commonly occurs through endocytosis with the cargo delivered to endosomes. These peptides were initially identified as part of larger proteins that were found to efficiently enter cultured cells (e.g., HIV-1 trans-activating activator of transcription [Tat] protein). The peptides are nontoxic, even at high concentrations, and typically too small to elicit an immune response. CPPs have been used to deliver peptides, proteins, oligonucleotides, plasmid DNA, and nanoparticles to the interior of cells. TAT and penetratin (both arginine-rich CPPs) have been used successfully increase cellular uptake of 75 and 200 nm liposomes. CPPs, e.g., low molecular weight protamine, have been shown to transport proteins across the cell membrane and into RBCs.

CPPs that may be used in the present invention include without limitation: (Arg)9 and various labeled and modified versions thereof, [Cys58]105Y, Cell Penetrating Peptide, α1-antitrypsin (358-374), Aminopeptidase N Ligand (CD13), NGR peptide, Antennapedia Leader Peptide (CT), Anti-BetaGamma (MPS-Phosducin-like protein C terminus), Bcl-2 Binding Peptide,Beclin-1, Buforin, Chimeric Rabies Virus Glycoprotein Fragment (RVG-9R), Antennapedia Peptide and various modifications thereof, hCT (Calcitonin), Hel 13-5, KALA, MAP, low molecular weight protamine, Mastoparan, Maurocalcine, MEK1 Derived Peptide Inhibitor 1, “Membrane Permeable Sequence” (MPS), MPGΔNLS, NGR Peptides 1, 2 and 3, NRTN (Neurturin),P1 (Human), or in, Pep-1-Cysteamine, Pep-1, pVEC (Cadherin-5), Rabies Virus Glycoprotein (RVG), SV-40 Large T, SV40 T-Ag, HIV-1TAT peptide and various modifications and fusion thereof, TfR Targeting Peptide, Transdermal Peptide, Transportan, etc.

In some embodiments, the cell penetrating moiety is a CPP such as penetratin or transportan.

The targeting and penetration moieties may be attached directly to reactive groups present on the polymers/copolymers that surround the agent of interest. For example, they may be attached by covalent bonds such as disulfide, esters, amides, etc. In other aspect, these moieties are attached to the polymeric matrix indirectly, e.g. via a linker or spacer molecule that is associated with one or more reactive groups present on the polymers/copolymers. Generally, such spacers or linkers are of a chain length ranging from about 5 to about 50 atoms, e.g. from about 10 to about 25 atoms, and have one end attached (e.g. covalently bonded) to a polymer reactive group and the other end attached (e.g. covalently or strongly non-covalently bonded) to a targeting or penetrating moiety. Exemplary linking groups include but are not limited to: positively charged peptides, negatively charged polypeptides, hydrophobic or hydrophilic polypeptides, depending on the nature of the interaction between targeting moiety, nanoparticle and cellular target.

According to one embodiment, the NPs are formed from grafted copolymers of PLL and PEG, the active agent that is encapsulated is BChE, and a single chain antibody, a monoclonal antibody, or a high affinity peptide with affinity for the target site membrane protein. Monoclonal against mouse glycophorin A, scFv Ter-119, or ERY1 peptide is used to target the nanoparticles to the RBC surface on mouse cells, while their analogs for binding to human glyocophorin A are used to target the RBC surface on human cells. Other embodiments utilize cell-penetrating peptides (CPPs) such as low molecular weight protamine to aid uptake or internalization of the NPs into the RBC.

Because the NPs described herein function in part by binding to RBCs, administration is by any method by which the NPs eventually enter the bloodsteam, e.g. parenterally such as intramuscular, intravenous or intranasal. The quantity of NP device that is administered will vary depending on the specific activity of the active agent, final concentration of the nanoparticles, etc. Currently, it is estimated that 2.5 ml of 100-fold concentrated BChE-containing nanoparticles (using 100 kDa ultrafiltration) would be sufficient to block a lethal dose of the organophosphate paraoxon (estimated based on specific activity of the current NP and in vitro sequestration assays with 40 nM paraoxon). We have confirmed that BChE-containing nanoparticles can be easily concentrated using cross-flow ultrafiltration. Those of skill in the art will recognize that preclinical in vivo studies are carried out to refine estimates for efficacy. Administration of the nanoparticle to a subject may be accomplished in any manner that permits contact between the nanoparticles and RBCs of the subject so as to result in binding of the at least one nanoparticle to an RBC (mediated by the targeting moiety) and, optionally, subsequent entry of at the least one nanoparticle into the RBC to which it is bound (mediated by the penetrating moiety), thereby loading the RBC with the agents that are associated with the nanoparticle. One or more than one type of NP may be administered to a subject

The present invention also provides compositions for use in treating a subject in need of or who may benefit from prophylactic administration of the nanodevices described herein. The compositions include one or more substantially purified NPs as described herein and a pharmacologically suitable carrier. The preparation of such compositions is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The liquids may be aqueous or oil-based suspensions or solutions. The active ingredients may be mixed with excipients which are pharmaceutically acceptable and compatible with the active ingredients, e.g. pharmaceutically acceptable salts. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The composition of the present invention may contain any such additional ingredients so as to provide the composition in a form suitable for administration. The final amount of NP in the formulations may vary. However, in general, the amount will be from about 1-99%. Still other suitable formulations for use in the present invention can be found, for example in Remington's Pharmaceutical Sciences, Philadelphia, Pa., 19th ed. (1995).

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

A subject in need of the NPs described herein is one who can benefit from exposure or contact with, and especially to slow exposure/contact over time (e.g. days, weeks or months), to the agent that is encapsulated within the NPs. The subject may be in need of treatment after the fact (the subject already has a disease or condition) or prophylactically (the subject has already developed or is likely to develop the disease or condition). In one aspect, the condition is that a subject has been exposed to OP, or is likely to be exposed to OP, and is treated with NPs comprising BChE. While in some instances, treatment may completely alleviate adverse, unwanted symptoms associated with the disease/condition, those of skill in the art will recognize that much benefit can accrue if symptoms are not completely eradicated but lessened, or the duration of symptoms is shortened, etc. Administration is generally to a mammal, for example a human. However, veterinary applications of this technology are also contemplated. Any subject with circulating RBCs may be treated using the present technology, modified to incorporate species-specific targeting ligands.

While generally a subject's own RBCs are utilized in vivo as described herein, without the need of removing and re-infusing the RBCs, the invention also encompasses removing of RBCs, treating (loading) of the RBCs as described herein, and administration of the RBCs. The RBCs may be autologous and readministered to the subject who is being treated and from whom they were taken, or they may be obtained from a donor and administered to a recipient in need.

### Applications of the Technology

Aspects of the invention address protection of soldiers, first responders and others from the toxic consequences of organophosphorus nerve agents (OPs). The OPs are among the most toxic of synthetic chemicals, and have been used in both chemical warfare and chemical terrorism. There is continuing concern that this type of weapon may be used (e.g., there has been evidence of their recent use in Syria). Drug therapy that treats some signs and symptoms of acute OP intoxication by protecting the “target” enzyme (i.e., acetylcholinesterase) in tissues from binding and inactivation by the OP is the most neuroprotective strategy currently known. The methodology described herein addresses the two main difficulties in the application of scavenger enzymes for protection against OP intoxication: the need for large amounts of purified enzyme and its rapid clearance from circulation once administered. Parenteral administration of BChE-encapsulated NPs that are affinity-targeted to and internalized by RBCs lead to prolonged circulation of the NPs, decreased clearance, and decreased demand for a large amount of the bioscavenger enzyme to exert the desired beneficial effect. Moreover, the invention is not limited to delivery of BChE, but also encompasses loading of circulating RBCs in situ with other drugs or macromolecules for long-term circulation.

The invention thus has a profound impact on chemical warfare agent science. Effective, practical approaches to protection against nerve agent intoxication have been problematic. This invention provides sustained, prolonged levels of bioscavengers in the circulation following a single administration. Attached, but in particular, internalized into circulating RBCs, BChE-nanoparticles can circulate for months, being shielded from proteolytic attack and immune surveillance.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is also to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### Examples

Parenteral administration of butyrylcholinesterase (BChE) is an effective prophylactic strategy to counteract organophosphorus anticholinesterase (OP) toxicity. Its efficacy is hampered however by rapid clearance of the enzymes from the circulation. Red blood cells (RBCs) can serve as carriers for drugs, peptides and enzymes. The Examples presented herein show that nanoparticles (NPs), for example cationic copolymer NPs, can be used to facilitate the delivery of agents of interest, for example, BChE, to RBCs. Prolonged circulation is achieved by combining RBC-targeting and cell-penetrating factors to recruit circulating RBCs as secondary carriers for long-term circulation. Successful implementation of such an approach “shields” bioscavenger enzyme molecules either attached to the RBC surface or following internalization into the RBCs. In either case, the enzymes then circulate for prolonged periods in the body, limited only by the life-span of the RBC and/or contact with other limiting factors such as contact with proteases. OP molecules interact with the bioscavenging enzymes in either orientation. Advantages of this approach compared to other prophylactic measures are the marked reduction in the need for re-treatment, and the use of less enzyme.

The various biocompatible components of the nanodevice break down in the body. The components are degraded along with their associated RBCs in the normal apoptotic pathways mediated by the reticuloendothelial system. If the NPs are internalized within RBCs, slow breakdown of the polymer and release of the free enzyme within the cytosol occurs. If the nanodevice does not enter into RBCs, NPs attached to the cell surface degrade over time, releasing the BChE molecules directly into the circulation.

Accordingly, in some aspects, PLL-g-PEO copolymers are used to encapsulate BChE and either a monoclonal or a single chain antibody to the erythroid cell-specific membrane protein glycophorin A (anti-GPA), or a short polypeptide with high affinity for glycophorin A allows selective targeting, while a cell-penetrating peptide (CPP) facilitates entry into RBCs. Once bound to/internalized into RBCs, the NPs circulate for prolonged times providing long-term protection.

### Example 1

### Generation and Characterization of Nanoparticles

This Example first describes the synthesis of grafted copolymers, the determination of polymer dimensions for optimized BChE encapsulation, and characterizes the physical properties and stability of selected NPs.

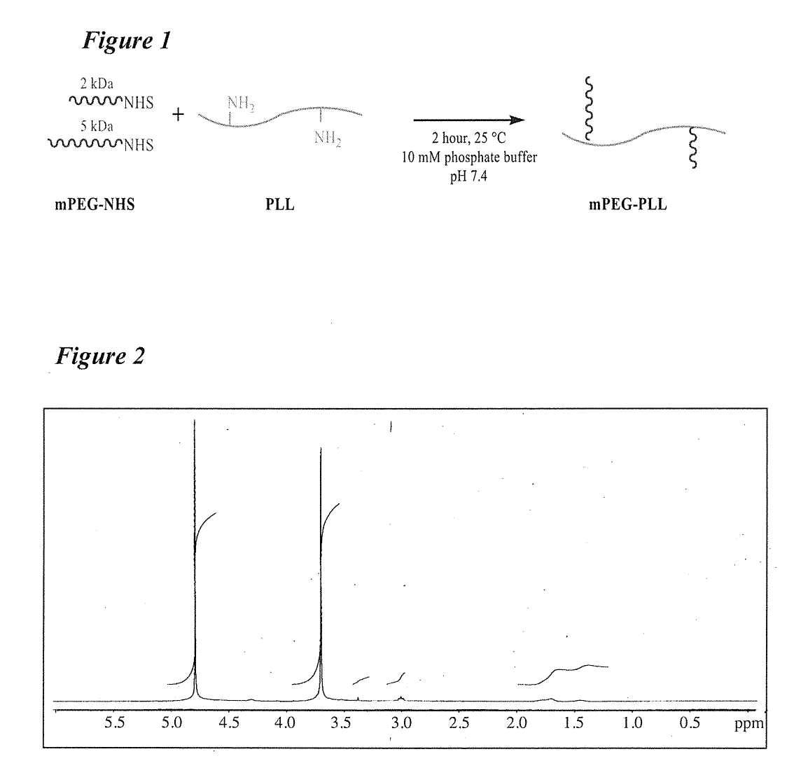

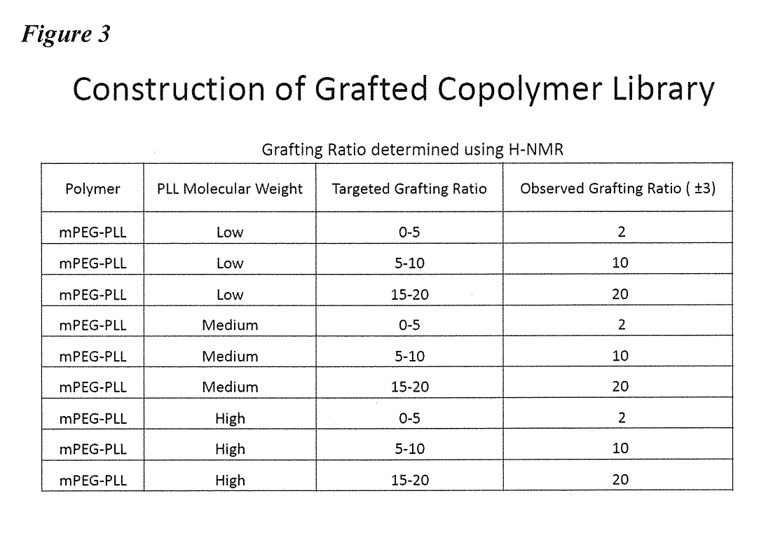

The goal was to synthesize a library of PEG-g-PLL polymers and determine the optimum polymer for encapsulating BChE. The grafted copolymers were composed of neutrally charged PEG grafted onto a cationic PLL backbone where the molecular weight of PLL and the grafting ratio of PEG to PLL were varied to optimize encapsulation of BChE. Factorial design using three molecular weights for PLL and three grafting ratios produced a library of nine unique PEG-g-PLL polymers. The polymer backbone, poly(l-lysine)•HBr, was purchased from Sigma with molecular weights of 4-15 kDa, 15-30 kDa, and 30-70 kDa. A 50/50 mixture of 2 and 5 kDa PEG was used to PEGylate the PLL backbone at grafting ratios of 2, 5, and 10 PEG per PLL. PEG was purchased from Creative PEGWorks (Winston Salem, N.C.) with an amine-reactive, n-hydroxysuccinimide esther (NHS) on the distal end. Synthetic conditions were selected to cover a range which would include a PEG-g-PLL polymer reported previously as complexing with BChE, improving bioavailability^{[18]}. PEG-NHS was conjugated to the PLL polymer through a reaction between the amine-reactive NHS group and the lysine ε-amino groups in PLL producing the product, PEG-g-PLL (FIG. 1, Step 1) as follows: a 50/50 mixture of 2 kDa and 5 kDa mPEG was reacted with poly(L-lysine) (PLL) polymers with molecular weight ranges 4-15 kDa, 15-30 kDa, and 30-70 kDa. The reactions were carried out at 25° C. for 2 hours in 10 mM phosphate buffer, pH 7.4. During reaction, NHS esters of the PEG react with amine groups along the polymer backbone, forming a grafted copolymer (mPEG-PLL). Un-reacted mPEG was removed using 10 kDa centrifugal concentrator and the product was dialyzed in PBS for 24 hours and then water for 48 hours. The grafted copolymer preparations were lyophilized for storage. FIG. 2 depicts exemplary results obtained when H¹ nuclear magnetic resonance (NMR) (400 MHz) was used to determine PEG grafting ratios (PEG:PLL). Peak areas were integrated. FIG. 3 shows an exemplary polymer library obtained and characterized in this manner.

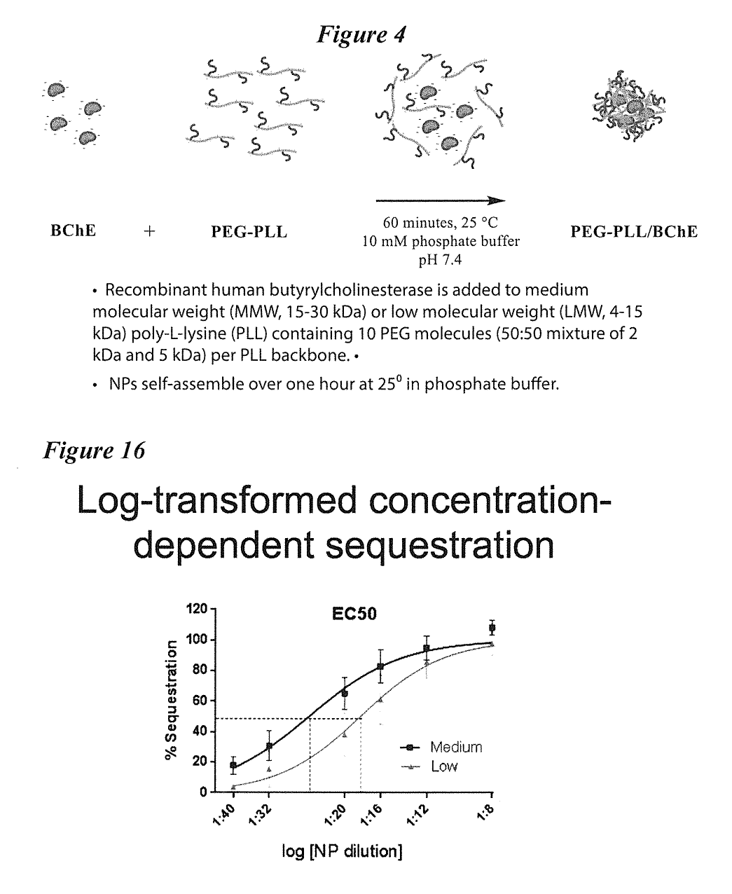

The grafted polymers were reacted with human recombinant BChE in order to generate nanoparticles (NPs) comprising the protein encapsulated in grafted copolymers. Experiments were conducted in which BChE was combined with medium molecular weight (15-30 kDa) or low molecular weight (4-15 kDa) poly-L-lysine (PLL), each of which contained 10 PEG molecules (50:50 mixture of 2 kDa and 5 kDa) per PLL backbone. the PEG-g-PLL/BChE (B-NPs or BChE-NPs) nanoparticles were formed by adding dropwise PEG-g-PLL (0.25 mg/ml of PBS) to BChE (0.50 mg/ml of PBS) while gently vortexing. The volume of each polymer added to BChE was adjusted, depending on the molecular weight of PLL and grafting ratio. Initially, volumes were calculated to produce a ratio of amino groups to carboxylic groups of Z_{+/-}=2, effective in past studies^{[18]}. BChE and the grafted copolymers were reacted for 60 minutes at 25° C. in 10 mM phosphate buffer, pH 7.4, during which time the NPs self-assemble. Formation of the B-NP occurs spontaneously due to electrostatic attraction between the positively charged PEG-g-PLL and the negatively charged BChE. After incubation at room temperature for 30 minutes, glutaraldehyde was added to cross-link the PLL polymer backbones and stabilize the complex. Cross-linking was performed at room temperature for 5 hours to ensure completion. The degree of cross-linking was controlled by varying the amount of glutaraldehyde added. Gaydess et al.^{[18]} demonstrated that 40% cross-linking stabilized the particles while retaining 80% of the enzyme activity. Following reaction, free BChE and polymer were removed from NPs using a 300 kDa MWCO centrifugal filter. Schematics of this procedure are presented in FIG. 4.

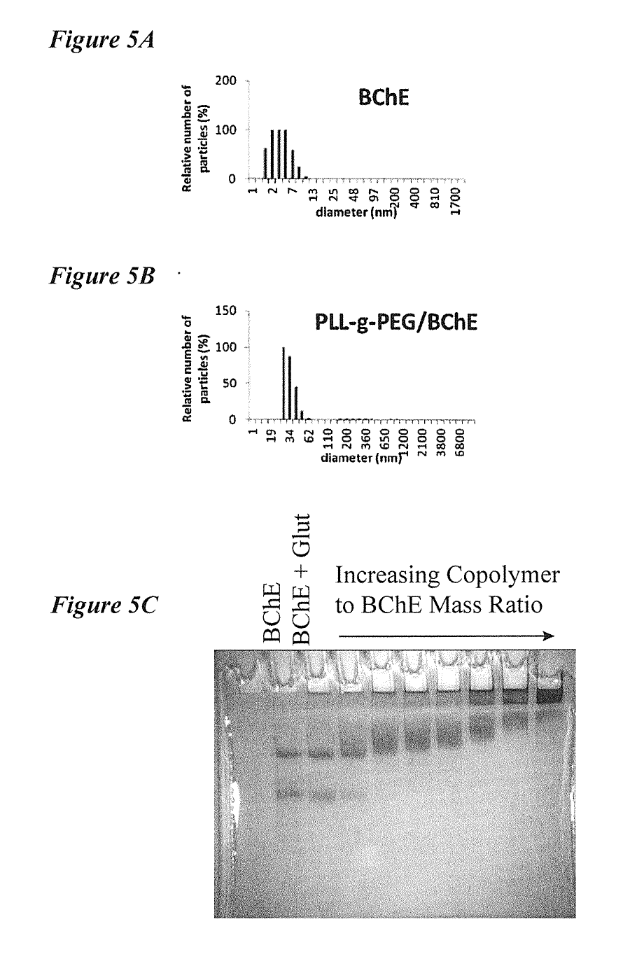

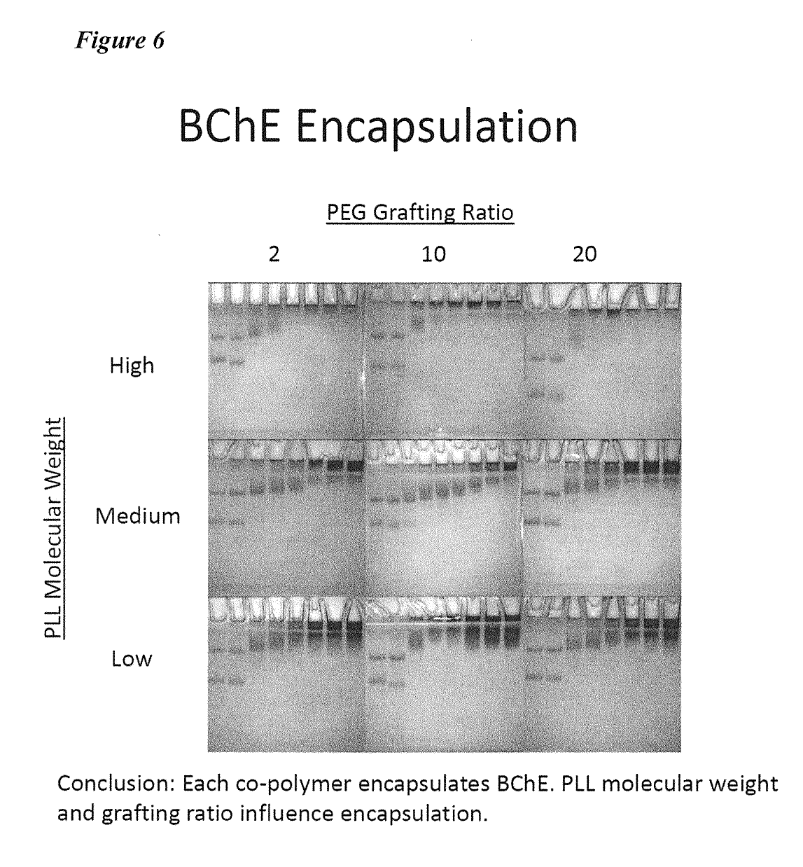

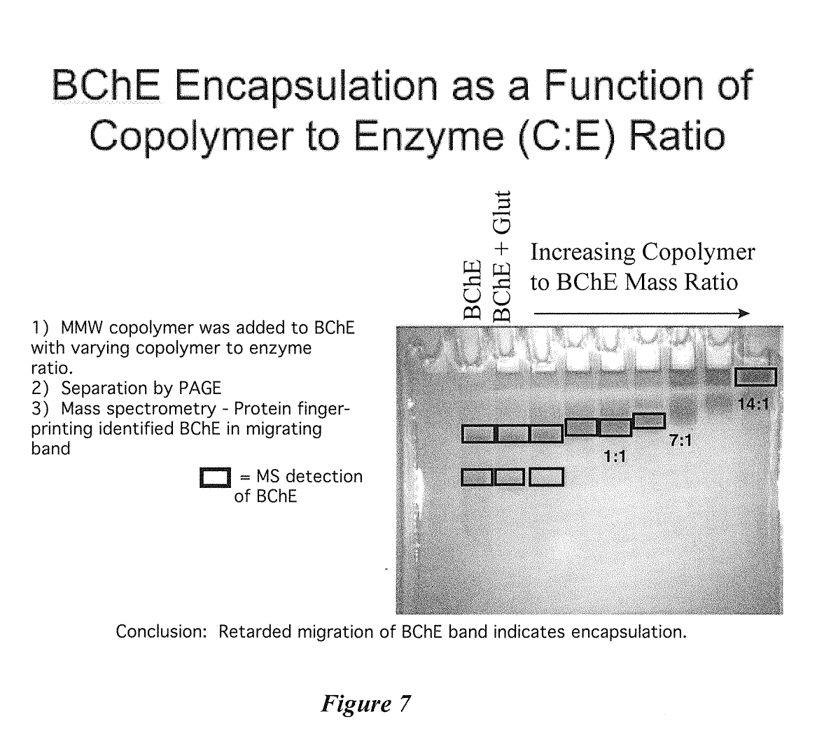

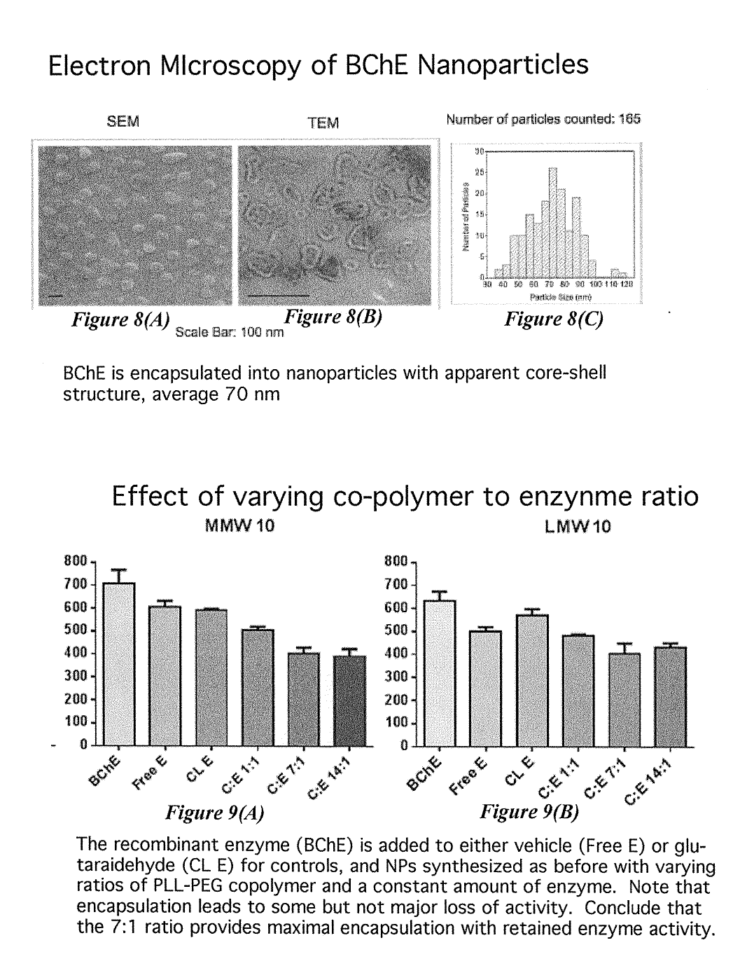

Particle synthesis was confirmed by dynamic light scattering (DLS) and polyacrylamide gel electrophoresis (PAGE), the results of which are presented in FIGS. 5A and B. Encapsulation efficiency was initially determined using non-denaturing polyacrylamide gel electrophoresis by loading B-NPs (30 μg BChE/well) onto a non-denaturing, 4-30% polyacrylamide gel and staining with Coomassie blue (FIG. 5C). Relative ratios of free BChE protein and the migration-retarded B-NPs were determined by densitometric image analysis. Encapsulation efficiency was determined by measuring the absorbance at 280 nm. FIG. 6 shows encapsulation of all possible nine co-polymer combinations. Note that in general as copolymer to enzyme ratio increased (from left to right on each gel), the protein stained bands migrated lesser into the gel, indicating larger size (encapsulation). FIG. 7 shows PAGE analysis of encapsulation with increasing copolymer: enzyme ratio, and mass spec fingerprinting of excised gel proteins. Note that human BChE was identified in the migration-retarded bands. FIG. 8A-C shows scanning and transmission electron micrographs of the NPs created using medium molecular weight, 10: grafting ratio, 7:1 copolymer:enzyme ratio. Note the NPs appeared to have core-shell structure, with average particle size of about 70 nm.

The method of Nostrandt et al.^{[48]} was used with butyrylthiocholine as substrate (1 mM final concentration) and a kinetic protocol to determine time-dependent substrate hydrolysis by BChE. FIG. 9 A and B shows BChE activity in different copolymer:enzyme fractions prepared with either medium molecular weight or low molecular weight copolymers, both with 10:1 grafting ratio. Reaction velocities were evaluated based on linear rates of substrate hydrolysis and the results were used to determine conditions for measuring BChE enzyme activity in the B-NPs. In vitro inhibition of enzyme activity by the B-NPs was evaluated and compared to concentration dependent inhibition of the non-encapsulated enzyme. IC₅₀ values for paraoxon against free and nano-complexed enzyme were compared to estimate the relative sensitivity to OP inhibition and any effect of nano-encapsulation, as described below.

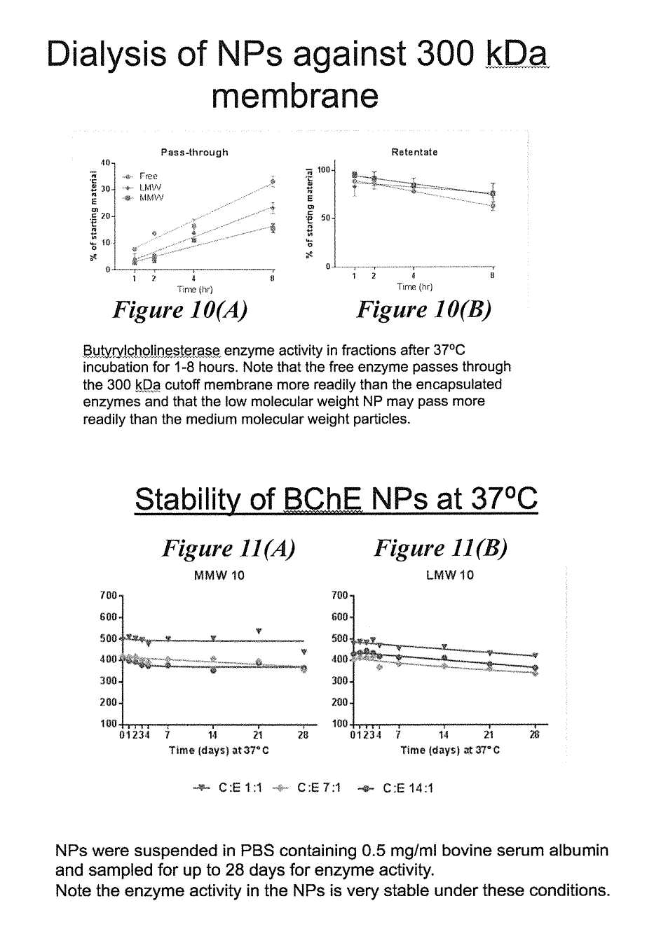

FIG. 10 A and B shows dialysis of NPs from low molecular weight and medium molecular weight polymer, grafting ratio 1:0, when dialyzed for 8 hours across a 300 kDa membrane in PBS containing 0.5 mg/ml BSA. Note that the free enzyme passes across the membrane more readily than the low molecular weight polymer particles, which also pass more readily than the medium molecular weight nanoparticles.

The stability of the 10:1 ratio copolymer (low and medium molecular weight, 7:1 copolymer to enzyme ratio) nanoparticles with time during incubation at 37° C. in PBS containing 0.5 mg/ml BSA was assessed. The results, which are presented in FIG. 11A and B, show that these are highly stable at 37° C.

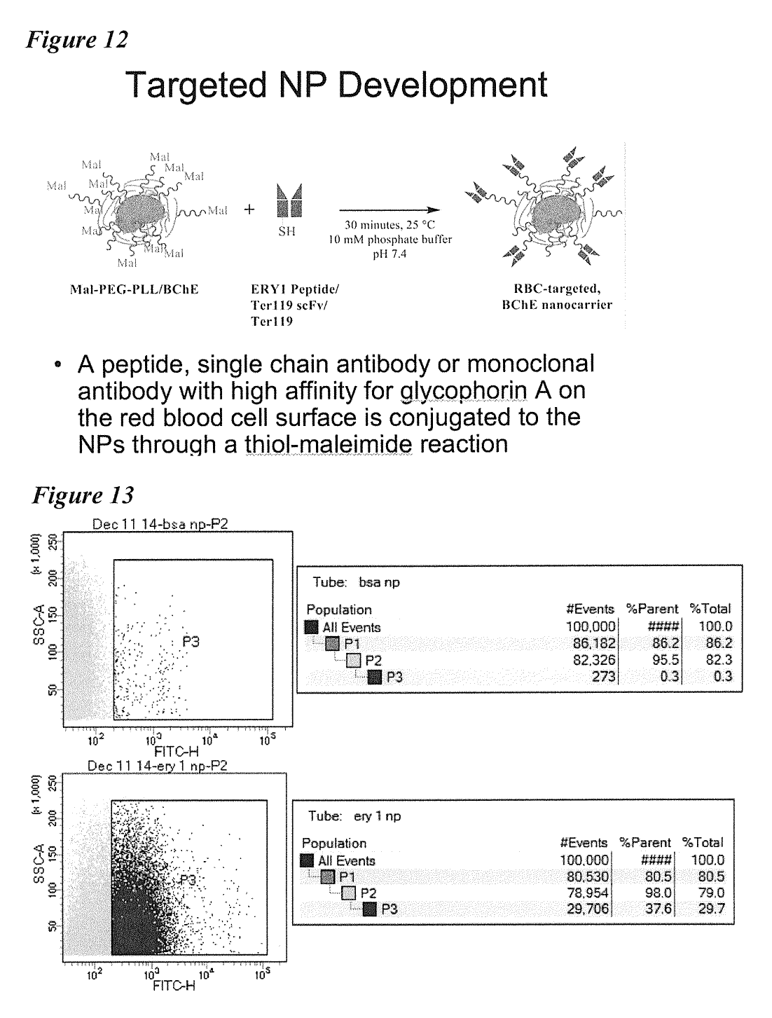

FIG. 12 shows the scheme for synthesis of the RBC-targeted NP for preclinical studies in mice. The targeting ligand is conjugated with maleimide groups on the terminal ends of PEG residues.

FIG. 13 shows flow cytometry evaluation of binding of the ERY1-conjugated, medium molecular weight, 10:1 grafting ratio, 7:1 copolymer:enzyme ratio nanoparticles to mouse red blood cells in vitro. These NPs were synthesized as before except that FITC-labeled bovine serum albumin was used in place of BChE. Note that the non-targeting (i.e., without ERY1) NPs had much less evidence of binding to the cells (as evidenced by less fluorescence intensity) compared to the ERY1-targeted NPs.

The ability of the organophosphate paraoxon to interact with both free and polymer encapsulated BChE was assessed. The experiment was performed as follows: non-encapsulated and encapsulated BChE fractions were allowed to incubate with varying concentrations of paraoxon for 20 minutes at 37° C. Substrate (butyrythiocholine, 1 mM) ws then added and residual BChE enzyme activity was measured. The results are shown in FIG. 14 A and B (top and bottom figure shows NPs made with medium molecular weight and low molecular weight, both with 10:1 grafting ratios, respectively). Both figures include free enzyme, crosslinked enzyme and the various copolymer:enzyme ratio fractions (1:1, 7:1, and 14:1) for each prep. As can be seen, free BChE, crosslinked BChE and BChE in various NPs (from low to high/copolymer to enzyme ratio) were similarly inhibited in a concentration-dependent manner by paraoxon. This shows that the OP binding site of BChE is available in the nanoparticles and appears similar in sensitivity to the inhibitor.

### Example 3

### OP Sequestration Assays

OP sequestration assays were carried out to determine the ability of BChE NPs to inactivate/sequester OP molecules. The reasoning behind the assay is as follows: It is known that OPs bind stoichiometrically to both BChE and another esterase, carboxylesteras (CarbE). If BChE-NP binds to paraoxon molecules during a pre-incubation step, then OPs in the reaction mixture will not be free to bind to and inhibit CarbE when added later. The assay was carried out as follows:

Step 1: A range of dilutions of BChE NPs was added to a solution of paraoxon (40 nM final concentration). Note that we determined the IC₅₀ for CarbE under these assay conditions was 7 nM paraoxon. The reaction mixtures were pre-incubated for 20 minutes at 37° C., and then pig liver CarbE was added. After another 20 minutes at 37° C., the BChE inhibitor ethopropazine (10 uM final) and the CarbE substrate p-nitrophenyl acetate (3 mg/ml final) was added. Residual CarbE activity was then measured for 3 minutes as an indirect indicator of NP-mediated inactivation of paraoxon.

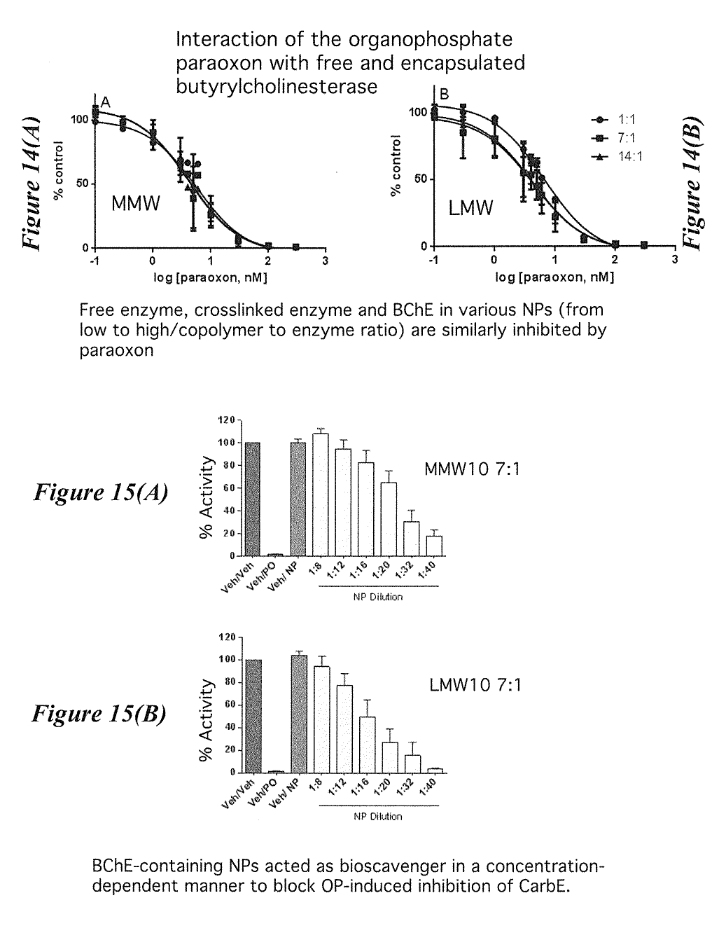

The results are presented in FIG. 15A and B (top and bottom figures show relative inactivation by medium and low molecular weight NPs, both 10:1 grafting ratios, respectively). As can be seen, with both the MMW 10 7:1 NPs and the LMW 10 7:1 NPS, BChE-containing NPs acted as bioscavengers in a concentration-dependent manner to block OP-induced inhibition of the CarbE.

### Example 4

### Additional Sequestration Analyses

FIG. 16 shows the data for these sequestration assays plotted as a non-linear function of NP dilution vs % protection from CarbE inhibition (% sequestration). Effective concentrations can be estimated (e.g., EC₅₀, the concentration that leads to 50% protection) as a measure of relative sequestration of different NPs Together, these assays further confirm the efficiency of encapsulation of human BChE into NPs and their resultant enzymatic and bioscavenging activities.

### Example 5

### Targeted NP Development

One goal of the present invention is the development of NPs that comprise moieties that target and bind to RBCs. Binding of such moieties to an RBC attach the NP to the surface of the RBC where it can exert its activity on molecules in the environment as the RBC circulates, or where it can then be internalized (taken up) into the RBC as described elsewhere herein. A schematic illustration of the production of an RBC-targeted BChE nanocarrier is presented in FIG. 12. According to some aspects, a RBC targeting molecule such as a peptide (e.g. ERY1, Ter119 scFv or Ter119), or a single chain antibody or monoclonal antibody with high affinity for glycophorin A (on the red blood cell surface) is conjugated to NPs through a thiol-maleimide (Mal) reaction for preclinical studies in mice, and later practical applications using human analogs of the targeting ligands.

### Example 6

### Further Investigations

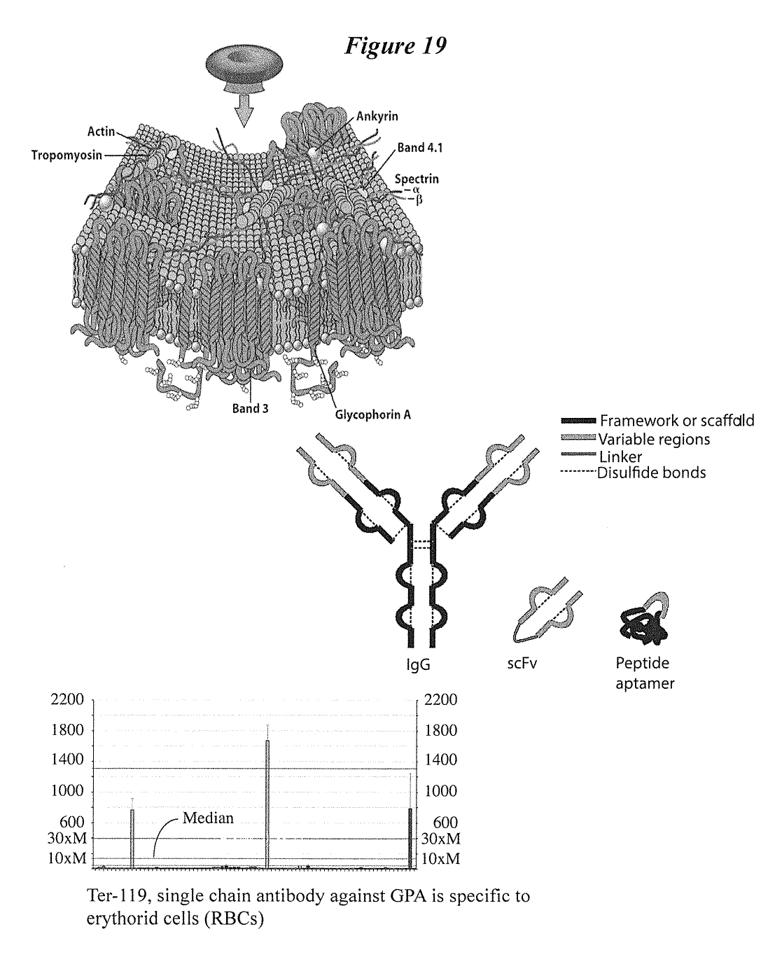

B-NPs equipped with protein modules that target them to RBC can be used to provide enhanced prophylaxis against OP toxicity. TER-119 (FIG. 19) was shown to selectively label mouse RBCs and their progenitors by high affinity binding to the erythroid cell surface antigen glycophorin A^{[54]}. scFv Ter-119 is used for coupling to B-NPs. Protein engineering is used to modify the original scFv Ter-119 to improve its targeting activity. These new scFv modules are expressed, purified and characterized re their stabilities and targeting efficiencies.

Express and Purify the Current scFv Ter-119 Single-Chain Antibody Against Glycophorin A.

Although the scFv Ter-119 binds the RBC, it has only been used as a fusion module in chimeric proteins. In the present aspect, scFv Ter-119 is used as a modular NP component. To produce the scFv Ter-119 polypeptide, scFv Ter-119 sequence is equipped with the 22-residue pelB prokaryotic signal for secretion^{[55]} and a C-terminal His6 sequence to enhance expression in E. coli. To facilitate development of binding assays for the detailed characterizations of our NP, fuse scFv ter-119 is also fused directly to gene sequences encoding commercially available fluorescent proteins. Periplasmic scFv Ter-119 constructs are induced in E. coli, purified by IMAC nickel affinity column chromatography, and further purified by ion exchange chromatography and gel filtration. Plasmid DNA encoding scFv Ter-119 is made. Sequences for scFv Ter-119 are cloned into E. coli plasmid vectors, utilizing standard PCR techniques to engineer start codons, stop codons, and flanking restriction sites. Tags for secretion, affinity purification, and cleavage by the TEV protease are derived from vector sequences or added by PCR. All DNA sequences are confirmed by dideoxy DNA sequencing. Transformants bearing scFv Ter-119 expression plasmids are grown at 37° C. with Tet or Amp (as appropriate to maintain plasmid expression), bringing the cultures to an absorbance at 600 nm of approximately 1.0, then cooling to 20° C. scFv expression is induced by the addition of IPTG and induction confirmed by SDS-PAGE of lysates from induced vs. un-induced cultures, with identity confirmed by mass spectrometry. Inductions are scaled up and repeated. Cells are harvested 20 hours post-induction, re-suspended, and slowly diluted further with osmotic shock buffer (40% sucrose, 0.033 M Tris-HCl, pH 7.3, 1.5 mM EDTA). After 10 min at room temperature, cells are harvested and the pellet is re-suspended in 1 ml of ice cold water containing MgCl₂. After 10 min on ice, the insoluble fraction is removed by centrifugation and the soluble fraction is collected. Solubility and secretion are assessed by SDS-PAGE of pellet vs. supernatant fractions from induced and un-induced cultures. scFv Ter-119 is dialyzed into loading buffer for Ni-NTA chromatography and eluted with an imidazole gradient. Peak fractions are identified using SDS-PAGE, pooled, and recombinant His and pelB tags removed by incubation of the protein with TEV protease (TEV:IL-1β 1:15 w:w), followed by passing back though Ni-NTA resin. ScFv-containing flow-through is concentrated by ion exchange, and subjected to final purification on a Superdex 75 16/60 gel filtration column using 500 mM sodium chloride, 25 mM Tris pH 7.4, 0.02% sodium azide buffer. Peak fractions are supplemented with 50% glycerol and stored at −80° C. Using the final purification scheme, scale up (10 L of E. coli) produces purified fractions pooled as “lots,” then is re-aliquoted and frozen (−80° C.). Two vials are characterized by protein concentration and purity (SDS-PAGE, mass spectrometry). scFv Ter-119 is also coupled to streptavidin using disuccinimidyl suberate, followed by desalting. Validated lots of scFv Ter-119 and its conjugates are used for coupling to BChE-NPs. In addition, the use of tandem scFv Ter-119 may increase GPA interactions within close proximity to each other, thus greatly enhancing NP binding to RBC. Methods are essentially the same as above. To facilitate assays of scFv Ter-119 localization in vivo, scFv Ter-119 is radioiodinated by the procedure of Coenen^{[56]} and used for synthesis of [¹³¹I]T-B-NPs for in vivo studies.

Synthesis and Evaluation of Transportan-Containing T-B-NPs.

The procedures above are repeated using the non-arginine-rich cell-penetrating peptide (CPP), transportan. Binding and internalization of [¹³¹I] scFv Ter-119/transportan-containing T-B-NPs is evaluated as above. The DThe [¹³¹I] scFv Ter-119 ligand and CPP are conjugated to the B-NPs. The resulting T-B-NPs are evaluated for binding and uptake. Radioiodinated T-B-NPs are synthesized as described above.

In Vitro Enzyme and OP Sequestration.

BChE enzyme activity and paraoxon sequestration of T-B-NPs are compared, as described above. Immunolocalization studies supplement activity-based studies above to confirm cellular and intracellular localization of human BChE, e.g. using anti-BChE.

Physicochemical Characterization and In Vitro Stability of T-B-NPs.

Particle size and surface charge are evaluated by dynamic light scattering and zeta-potential measurements, performed to ensure sizes below 300 nm and a negative or near-neutral zeta-potential. Particles of approximately 15 nm^{[18]} are detected, well below the 300 nm threshold concern for edema. The surface charge is not positive, which could lead to aggregation. Transmission electron microscopy (TEM) reveals particle morphology. Particle size and zeta-potential are used to estimate in vitro stability. Neutrally charged particles self-aggregate at high concentrations, shortening shelf-life. Particle size is measured at regular intervals for signs of aggregation (increasing average size) or dissociation of polymer/protein complex is (average particle size decreasing). Samples are tested after storage 4, 25 and 37° C. Batches of stored T-B-NPs are evaluated at times throughout the project by enzyme activity assay and OP sequestration.

Binding, Localization and Internalization of T-B-NPs to Mouse RBCs In Vitro Using Confocal Microscopy.

The ability of fluorescent T-B-NPs to bind and be internalized by RBCs in vitro is assessed using fluorescence labeling of BChE with IRDye 800CW. Briefly, the N-hydroxy-succinimide ester reactive group of the dye is stably conjugated with a primary amine of BChE and purified. Binding/internalization of T-B-NPs to mouse RBCs compared to binding/internalization the non-encapsulated enzyme is determined. Viability of mouse RBCs (e.g. osmotic fragility, etc.) with attached/internalized NPs is evaluated.

Comparative Pharmacokinetics of BChE, Histopathology and Immune Alterations after Intramuscular Administration of T-B-NPs Vs. Equivalent Amounts of Non-Encapsulated BChE are Assessed

The ability of the T-B-NPs to bind RBCs in vivo, enhance circulation half-life, and sequester OP molecules ex vivo is assessed. Free enzyme (>90%) following parenteral administration is typically cleared from the circulation over about ˜200 hours (half-life in mouse=67 hour)^{[58]}. Prophylaxis is enhanced when a maximal amount of targeted NPs have localized with circulating RBCs. A mouse model is used to determine clearance and duration of T-B-NPs circulation. C57B1/6n mice (n=6/treatment/time-point) are injected (im) into the thigh muscle with either vehicle, B-NPs (as targeting control), T-B-NPs or an equivalent amount of free rBChE (about 700 BChE units/gram body weight^{[58]}. Blood is collected and used for enzyme/OP sequestration assays. Plasma, RBC and tissue BChE is evaluated using the standard spectrophotometric method using butyrylthiocholine as substrate^{[48]}. Parallel assays will be performed with a cell-permeant BChE substrate, [³H]cocaine, by the method of Brimijoin et al.,^{[64]}. This substrate easily crosses membranes and is efficiently hydrolyzed by BChE (but not AChE), thus it is effective in measuring BChE inside RBCs without “contamination” from AChE.

BChE Antibody-Based Immunostaining Approaches.

Anti-BChE mouse monoclonal antibody is used to confirm cellular and intracellular localization of BChE in mice, e.g. in skeletal muscle, liver, spleen, kidney, lung, heart, and brain tissue. Selective immunostaining with anti-BChE enables detection of human BChE in tissues without interference from mouse BChE^{[66]} and also specifically detects BChE against a high background of AChE (e.g. at neuromuscular junctions). The results provide strong validation of the red-cell targeting peptide transfer.

Evaluate in vivo toxicity of T-B-NPs.

The present nanodelivery devices are biocompatible. Histopathology is evaluated however in skeletal muscle, liver, spleen, kidney, lung, heart, and brain tissues

Evaluate Biodistribution of T-B-NPs Using Live Mice.

A major project goal is to achieve long-term circulation of BChE. Mice are tracked over time to determine T-B-NPs biodistribution. Groups (n=6) are injected through the tail vein with 100 μl of rBChE, B-NPs, or T-B-NPs containing near-infrared fluorescent dye-labeled rBChE, with two saline-injected mice for background controls. The mice are imaged (30 min-24 h), then daily until no signal is detected.

Determine Effects of In Vivo T-B-NPs on Mouse RBC Viability, Morphology and Osmotic Fragility Ex Vivo.

Aliquots of mouse blood cells are used to evaluate RBC parameters ex vivo. RBCs are collected and viability, morphology and osmotic fragility are evaluated.

Paraoxon Toxicity Studies to Determine Sublethal, Lethal and Supralethal Exposure Levels.

C57B1/6n mice (n=4/treatment group) are given one of a range of dosages (3, 3.9, 5.1, 6.6, 8.6 or 11.2 mg/kg) of paraoxon (10 ml/kg, sc in ice cold saline) and functional signs/lethality measured over 24 hours. A dose that elicits 1) signs of toxicity but no lethality (toxic), 2) 1-3 deaths (lethal) and 3) complete lethality (supralethal) are selected for further studies.

Evaluate Modulation of Dose-Dependent OP Toxicity in Mice Pre-Treated with Vehicle, T-B-NPs, or an Equivalent Amount of Non-Encapsulated rBChE.

Mice (n=8/treatment group) are pre-treated (im) with either vehicle, one dose of T-B-NPs (to yield maximal peak BChE concentration), or an equivalent dose of free BChE and then challenged with either vehicle or toxic, lethal or supralethal dosages of paraoxon. The timing of OP dosing is based on the time to reach peak BChE levels (Tₘₐₓ) following either T-B-NPs or free enzyme, with treatment in both cases coinciding with peak BChE. Vehicle controls are challenged with OP. Mice are evaluated for toxicity as described^{[53]} from 0.5-24 hours. Blood pressure, heart rate, blood flow and blood volume are measured in subsets using a non-invasive tail cuff monitoring system. Brain, heart, skeletal muscle, lung and whole blood are collected. Blood is separated into RBC and plasma fractions and hematocrit is determined, along with evaluation of RBC membrane blebbing and cell condensation for evidence of eryptosis. All tissues are analyzes for AChE and BChE activities as described above. Prophylaxis in T-B-NPs vs free enzyme groups (relative to vehicle controls) is compared and comparative efficacy of the T-B-NPs to protect against OP toxicity is determined.

Comparative Time-Dependent Modulation of OP Toxicity by Vehicle, T-B-NPs or Equivalent Amount of Non-Encapsulated BChE.

Mice (n=8/treatment group) will be pretreated with vehicle, T-B-NPs or an equivalent amount of free BChE. OP (supralethal dose) is administered at the respective Tₘₐₓ for free and encapsulated BChE, and at two other time-points (e.g., 3× Tₘₐₓ; 6× Tₘₐₓ). Functional toxicity and lethality is recorded and extent of protection in T-B-NPs vs free enzyme groups is compared. Blood pressure, blood flow, heart rate and blood volume are measured.

In these examples, a nanodevice-mediated prophylactic effect against OP toxicity is demonstrated with enhanced circulation, lesser bioscavenger enzyme requirement, and higher efficacy compared to free enzyme.
[1] Pope, C N. (2010). Toxicology of Organophosphorus and Carbamate Compounds (R C Gupta, ed), Academic Press, San Diego, Calif., pp. 271-292.
[2] Weissman B A, Raveh L. (2011). Toxicology 290: 149-55.
[3] Dirnhuber P, et al., (1979). J Pharm Pharmacol. 31: 295-9.
[4] Heyl W C et al., (1980). Drug Chem Toxicol. 3:319-32.
[5] French M C et al., (1979). J Pharm Pharmacol. 31:290-4.
[6] Kassa J et al., (2012). Basic Clin Pharmacol Toxicol. 110:322-6.
[7] Doctor B P et al., (1991). Neurosci Biobehav Rev. 15: 23-8.
[8] Raveh L et al., (1993). Biochem Pharmacol. 45:2465-74.
[9] Saxena A, Sun W, Luo C, Doctor B P. (2005). Chem Biol Interact. 157-158:199-203.
[10] Doctor B P, Saxena A. (2005). Chem Biol Interact. 157-158:167-71.
[11] Saxena A et al., (2006). J Mol Neurosci. 30:145-8.
[12] Saxena A et al., (2011). Biochem Pharmacol. 81:164-9.
[13] Myers T M et al., (2012). Neurotoxicol Teratol. 34:323-30.
[14] Buske J et al., (2012). Eur J Pharmaceut Biopharmaceut: 81:57-63.
[15] Santander-Ortega, M N et al., (2010). Colloid Polymer Sci 288:141-150.
[16] Kochendoerfer G. (2003). Expert Opin Biol Ther. 3:1253-61.
[17] Aliabadi et al., (2005). Biomaterials 26:7251-7259.
[18] Gaydess et al., (2010). Chem Biol Interact. 187:295-8.
[19] Millán C G et al., (2004). J Control Release. 95:27-49.
[20] Garratty G. (2008). Vox Sang. 94:87-95.
[21] Kwon Y M et al., (2009). J Control Release. 139:182-9.
[22] Kravtzoff R et al., (1990). J Pharm Pharmacol. 42:473-6.
[23] Graham M L. (2003). Adv Drug Deliv Rev. 55:1293-302.
[24] Telen M J (2000). Semin. Hematol. 37:130-142.
[25] Wautier J L, Wautier M P. (2004). Clin Hemorheol Microcirc. 30:181-4.
[26] Oliveira S, Saldanha C. (2010). Clin. Hemorheol. Microcirc. 44:63-74.
[27] Oliveira S et al., (2012). Biochim Biophys Acta. 18481-90.
[28] Moriyam R et al., (1993). J Biol Chem 268:10990-10996.
[29] Poole J. (2000). Blood Reviews 14:31-43.
[30] Bordin L et al., (2005). Mol Membrane Biol 22:411-420.
[31] Williamson R C, Toye A M. (2008). Blood Cells Mol Dis. 41:35-43.
[32] Karsten U et al., (2010). Int Immunopharmacol. 10:1354-60.
[33] Adekar S P et al., (2011). PLoS One 6(3): e17491.
[34] Taborelli A et al., (2011). Med Sci Law. 51 Suppl 1:S16-9.
[35] Spitzer et al., (2004). Mol Immunol. 40:911-9.
[36] Zaitsev S et al., (2010). J Pharmacol Exp Ther. 332:1022-31.

### [37] Taylor N D et al., (2010). Protein Eng Des Sel. 23:251-60.

### [38] Kontos S et al., (2013). Proc Natl Acad Sci USA. 110: E60-8.

[39] Zorko M, Langel U. (2005). Adv. Drug Deliv Rev. 57:529-45.

### [40] Lundberg P, Langel U. (2003). J Mol Recog 16:227-233.

### [41] Hawiger J. (1999). Curr Opin Chem Biol 3: 89-94.

### [42] Schwarze S R et al., (1999). Science 285:1569-72.

### [43] Astriab-Fisher A et al., (2002). Pharm Res. 19:744-54.

### [44] Tseng Y L et al., (2002). Mol Pharmacol. 62: 864-72.

### [45] Torchilin V P et al., (2001). Proc Natl Acad Sci USA. 98: 8786-91.

### [46] Chung, H. S. (2010). Doctor of Philosophy Disseration, University of Michigan.

### [47] Sun H et al., (2002). Mol Pharmacol 62: 220-224.

### [48] Nostrandt A C et al., (1993). Fundam Appl Toxicol. 21:196-203.

### [49] Hosea N A et al., (1995). Biochemistry 34:11528-11536.

### [50] Radic Z et al., (1993). Biochemistry 32: 12074-12084.

### [51] Mintz K P, Brimijoin S. (1985). J Neurochem 44: 225-232.

### [52] Brimijoin S et al., (1983). Mol Pharmacol 24: 513-520

### [53] Karanth S et al., (2001). Toxicol Appl Pharmacol. 177: 247-55.

### [54] Kina T et al., (2000). Br J Haematol. 109: 280-7.

### [55] Choi J H, Lee S Y. (2004). Appl Microbial Biotechnol. 64:625-35.

### [56] Coenen, H H et al., (1983). Radiochimica ACTA 34:47-68.

### [58] Duysen E G, Lockridge O. (2008). Chem Biol Interact 175:119-124.

[59] Terman D S et al., (2013). PLoS One, 8(1), e52543.

### [60] Zhao Y et al., (2011). ACS Nano 5:1366-1375.

### [61] Ranjan A et al., (2009). Antimicrob Agents Chemother, 53:3985-3988.

### [62] Ranjan A et al., (2010). Antimicrob Agents Chemother, 54:3524-3526.

### [63] Arimitsu N et al., (2003). Genes Cells 8:769-77.

### [64] Brimijoin, S et al., (2002). Anal Biochem 309:200-205.

### [65] Ranjan A et al., (2012). J Control Release, 158:487-494.

### [66] Gao, Y, Brimijoin S. (2009). J Pharmacol Exp Ther 330: 449-457.

[67] Johnson N D et al., (2009). Neurotoxicology 30:386-392.
[68] Jenkins D E et al., (2003). Clin Exp Metastasis 20:745-756.

What is claimed is:

1. A nanoparticle delivery device comprising
an agent of interest encapsulated in a polymeric matrix which is penetrable by a ligand of said agent of interest;
a red blood cell (RBC) targeting moiety; and, optionally
an RBC penetrating moiety.

2. The nanoparticle delivery device of claim 1, wherein said polymeric matrix is cationic.

3. The nanoparticle delivery device of claim 2, wherein said polymeric matrix comprises poly-L-lysine (PLL)-polyethylene glycol (PEG) grafted copolymers.

4. The nanoparticle delivery device of claim 1, wherein said agent of interest is selected from the group consisting of butyrylcholinesterase (BChE) and asparaginase.

5. The nanoparticle delivery device of claim 1, wherein said RBC penetrating moiety is a cell penetrating peptide (CPP).

6. The nanoparticle delivery device of claim 4, wherein said CPP is selected from the group consisting of Tat, penetratin, low molecular weight protamine, and transportan.

7. A method of delivering an agent of interest to a subject in need thereof, comprising
administering to said subject a therapeutically effective amount of a nanoparticle delivery device comprising
an agent of interest encapsulated in a polymeric matrix which is penetrable by a ligand of said agent of interest;
a red blood cell (RBC) targeting moiety; and, optionally
an RBC penetrating moiety.

8. The method of claim 7, wherein said polymeric matrix is cationic.

9. The method of claim 8, wherein said polymeric matrix comprises poly-L-lysine (PLL)-polyethylene glycol (PEG) grafted copolymers.

10. The method of claim 7, wherein said agent of interest is selected from the group consisting of butyrylcholinesterase (BChE) and asparaginase.

11. The method of claim 7, wherein said RBC penetrating moiety is a cell penetrating peptide (CPP).

12. The method of claim 11, wherein said CPP is selected from the group consisting of Tat, penetratin, low molecular weight protamine, and transportan.

13. The method of claim 7, wherein said method is performed to treat or prevent a disease or condition that is cured or ameliorated by said agent of interest.

14. The method of claim 7, wherein said agent of interest persists in or on RBCs of said subject for a sustained period of time.

15. The method of claim 14, wherein said sustained period of time is weeks or months.

16. A method of bioscavenging and unwanted substance from the circulatory system of a subject in need thereof, comprising
administering to said subject a nanoparticle delivery device comprising
an agent of interest encapsulated in a polymeric matrix which is penetrable by said unwanted substance;
a red blood cell (RBC) targeting moiety; and, optionally
an RBC penetrating moiety;
wherein said nanoparticle delivery device is delivered in a quantity sufficient to scavenge said unwanted substance from the circulatory system of said subject.

17. The method of claim 16, wherein said polymeric matrix is cationic.

18. The method of claim 16, wherein said polymeric matrix comprises poly-L-lysine (PLL)-polyethylene glycol (PEG) grafted copolymers.

19. The method of claim 16, wherein said RBC targeting moiety is selected from the group consisting of:

20. The method of claim 16, wherein said RBC penetrating moiety is a cell penetrating peptide (CPP).

21. The method of claim 20, wherein said CPP is selected from the group consisting of Tat, penetratin, low molecular weight protamine, and transportan.

22. The method of claim 16, wherein said agent of interest is BChE and said unwanted substance is an organophosphorus anticholinesterase.

23. The method of claim 16, wherein said agent of interest persists in or on RBCs of said subject for a sustained period of time.

24. The method of claim 23, wherein said sustained period of time is weeks or months.

25. A red blood cell comprising
a nanoparticle delivery device comprising
an agent of interest encapsulated in a polymeric matrix which is penetrable by a ligand of said agent of interest;
a red blood cell (RBC) targeting moiety; and, optionally
an RBC penetrating moiety.
